(19) United States
(12) Patent Application Publication
(10) Pub. No.: US 2009/0202978 A1
(43) Pub. Date: Aug. 13, 2009

(54) Method and apparatus for freezing of a biological material

(76) Inventor: Ginadi Shaham, Yavneh (IL)

Correspondence Address: THE NATH LAW GROUP, 112 South West Street, Alexandria, VA, 22314, US

(21) Appl. No.: 12/068,953

(22) Filed: Feb. 13, 2008

(51) Int. Cl. C12N 1/04; F25D 3/10; F25D 25/00

(52) U.S. Cl. 435/1.3; 62/465; 62/457.1; 62/120; 435/307.1

(57) ABSTRACT

The present invention provides a device for freezing biological material, comprising: a cooling unit; a heat insulation unit, having an interface with said cooling unit and a common longitudinal axis passing through said interface transversely thereto; an elongated slot disposed at least in said heat insulation unit and extending along said axis, said slot comprising a first slot portion remote from said interface and a second slot portion adjacent said interface; wherein said first and second slot portions have respective first and second dimensions measured perpendicularly to said slot's axis, the former being greater than the latter.

Further provided is a container for accommodating therein of a biological material, said container having a longitudinal container axis and comprising a first container portion having a first dimension and a second container portion having a second dimension smaller than the first dimension, both measured perpendicularly to said longitudinal container axis; the container portions being in fluid communication; said container being adapted for carrying said biological material in said first container portion, and a solution different from said material, fully filling said second container portion, which is essentially free of said biological material, and at least partially filling said first container portion so as to be in contact with said biological material; said container further comprising retention means for preventing passage of said biological material from said first container portion to said second container portion.

Methods for freezing biological material making use of the device and container of the invention is also part of the invention.

### FIELD OF THE INVENTION

This invention relates to a method and an apparatus for freezing of a biological material.

### LIST OF REFERENCES

The following references are considered to be pertinent for the purpose of understanding the background of the present invention:

1. WO06/016372;

2. U.S. Pat. No. 5,873,254;

3. WO 03/056919;

4. WO 03/020874;

5. U.S. Pat. No. 6,337,205 (Wisniewski);

6. U.S. Pat. No. 5,863,715 (Rajotte).

### BACKGROUND OF THE INVENTION

In freezing of biological material, two freezing stages are recognized: nucleation and crystallization. In the first stage ice nucleation occurs in the solution outside the cells. In order to minimize cellular damage, it is critical to control during this stage (nucleation) both the interface velocity of the cold front and the direction of thermal gradient within the object. Normally, in some biological materials (e.g. blood, cell suspensions, plasma, semen and other liquid samples) the best survival is obtained when the freezing rate at this stage is relatively rapid (10° C./min or more). In other cases (e.g. organs or organ fragments), it is accepted that a slow freezing rate at this stage (0.5° C./min or less) would improve freezing.

The next stage is that of crystallization, an exothermic process that produces latent heat within the frozen material, causing a period of time when the biological material remains isothermal, or even experiences an increase in temperature: latent heat exudes from the biological material and thus, although the material is being cooled no temperature change is observed or the temperature may even rise. This, in turn, causes spontaneous freezing and thawing cycles which are hazardous to the biological material.

Permitting osmosis of water out of the cells at this stage would reduce damage to the cells, and the increase of intracellular concentration would cause the cells to vitrify rather than freeze. This is affected by the rate of freezing, and thus, in order to optimize the biological material's survival of this stage control of the rate of freezing is important. The optimal rate depends on the type and composition of the biological material being frozen.

In addition to the above, cryopreservation of material having a large volume (e.g. tissues, organs or portions thereof) is associated with heat transfer and mass problems that are not associated to the same extent with cryopreservation of isolated cells. For example, in conventional freezing methods, ice grows at an uncontrolled velocity and morphology and may disrupt and kill cells by mechanical destruction of the tissue architecture. Due to the large size of macroscopic material, large uncontrolled thermal gradients may develop from the surface of the sample to its interior.

One method that was devised to allow freezing biological material is disclosed in WO2006/016372. In this publication, the temperature of a biological material to be frozen is changed from a first temperature to a second temperature within a time period, one of the said first or second temperature being above freezing temperature and the other being below freezing temperature. Freezing is achieved by placing the biological material in tight contact with at least one, preferably between two heat exchangers, and controlling the temperature in at least one of said heat exchangers such that a freezing temperature front propagates in said material away from at least one of the two heat exchangers.

A method of freezing biological material of a large volume is disclosed in U.S. Pat. No. 5,863,715. In this patent, the biological material is placed in a flexible container, such as a bag. The bag is then flattened in a holder that maintains an essentially constant cross-sectional area of the bag in order to minimize thermogradients. The holder is then cooled along with the bag contained therein.

It is well established that directional freezing, a process in which a cold front propagates in a controlled manner through the frozen object, improves the chances of biological material to survive freezing and thawing. In this process a temperature pattern (or gradient) is established in the object being frozen to form a propagation cold front within the object, resulting in improved chances of survival.

A successful method of directional freezing is disclosed in U.S. Pat. No. 5,873,254. In this patent, a freezing apparatus is used to establish a laterally varying thermal gradient and the biological sample is moved along the thermal gradient at a controlled velocity. Additional methods were developed in order to improve the freezing of large volume objects. For example, WO 03/056919 discloses freezing biological samples via an isothermal stage, wherein the temperature is changed until temperature of the sample in an outer zone equals intermediate temperature and changing temperature until the majority of the sample is in a final temperature. This method may be used in conjunction with directional freezing but is not limited thereto. Another process is disclosed in WO 03/020874 in which the biological sample is agitated during its migration along a thermal gradient.

A method for cryopreservation of a biological is disclosed in U.S. Pat. No. 6,337,205. The sample to be frozen is inserted into special oblong vials that have special appendages, termed “ice crystal-nucleating structures”, situated at the opposite ends of the vial's oblong cross-section. The vials are placed within a compartment of a cryopreservation apparatus, said compartment containing a cryopreservation fluid. A freezing front is then generated at one of the walls of the apparatus that is adjacent to one of the appendages, and propagates through the cryopreservation fluid. Due to the special shape of the appendage, nucleation begins at the appendage, and thus the cold front propagates within the sample in a direction that is away from the cooling wall and along the oblong cross section of the vial. In an alternative disclosed in U.S. Pat. No. 6,337,205, two cold fronts may be generated in the compartment, in opposing directions, by opposing walls of the apparatus. In this method, the freezing of the sample is achieved indirectly, in the sense that the cooling wall of the apparatus cools the cryopreservation fluid, which in turn cools the vial (and the sample within it).

### BRIEF DESCRIPTION OF THE DRAWINGS

In order to understand the invention and to see how it may be carried out in practice, embodiments will now be described, by way of non-limiting example only, with reference to the accompanying drawings, in which:

FIGS. 1A to 1C are schematic perspective cross sections of devices in accordance with two non-limiting examples of the invention;

FIGS. 2A to 2C are longitudinal cross-section views of a sample container in accordance with three examples of the invention;

FIGS. 3A and 3B are schematic illustrations of a pouch and a sealing portion according to the present invention; and

FIGS. 4A and 4B are schematic, perspective cross sections of the devices of FIGS. 1A to 1B respectively, while carrying the sample container of FIGS. 2A and 2B.

### SUMMARY OF THE INVENTION

In accordance with one aspect of the present invention there is provided a device for freezing biological material, comprising:
a cooling unit;
a heat insulation unit, having an interface with said cooling unit and a common longitudinal axis passing through said interface transversely thereto;
an elongated slot disposed at least in said heat insulation unit and extending along said axis, said slot comprising a first slot portion remote from said interface and a second slot portion adjacent said interface;
   wherein said first and second slot portions have respective first and second dimensions measured perpendicularly to said axis, the former being greater than the latter.

The slot within the device is adapted for receiving therein a container having a first container portion for accommodating said biological material and a second container portion free of said material, such that when the container is mounted within said slot, the first container portion is disposed in the first slot portion and a portion of the second container portion is within the second slot portion.

The term “free” when used in connection with biological material, means that essentially all the biological material needed to be viable after thawing is located away from the second container portion. “Essentially all” means that at least 80%, or at least 90% or even at least 95% and at times even all the biological material to be frozen is located away from the second container portion. In case the biological material contains many small members (small particulate matter such as cells, clusters of cells, microorganisms, tissue fragments for example bone chips, etc.) some of the small members may be found in the second container portion. Small members are members measuring no more than 5mm or even no more than 2 mm in any direction. In case the biological material comprises larger (e.g. measuring at least 2 mm, or even at least 5 mm in at least one direction) solid or semi-solid body of biological material (namely, non-fluid material, for example one or several slices of tissue or an osteochondral cylinder)) the term “free” may be taken to mean that the body of the biological material is absent (located away) from the second container portion, while free cells or other biological debris material may still be present in the second container portion.

The device may comprise one of more of the following features:
The device may comprise one or more elongated slots, each adapted for receiving therein a container.
The elongated slot in the heat insulation unit may be adapted for holding one or more containers.
The cooling unit may comprise a chamber for holding a cooling fluid medium.
The heat insulation unit may be fixed to the cooling unit.
The fluid medium may be a liquid with the heat insulation unit floating on top of the liquid.
The cooling unit may be in a form of a solid cooling block, said block comprising a recess which abuts said interface and is in fluid communication with said elongated slot disposed in said heat insulation unit. The recess of the block may be adapted for having thermal contact with the second container portion. The recess may be further adapted for accommodating a cooling liquid therein.

In accordance with another aspect of the present invention, there is provided a container for accommodating therein of a biological material, said container having a longitudinal container axis and comprising a first container portion having a first dimension and a second container portion having a second dimension smaller than the first dimension, both measured perpendicularly to said container axis; the container portions being in fluid communication; said container being adapted for carrying said biological material in said first container portion, and a solution different from said material, fully filling said second container portion, which is free of said biological material, and at least partially filling said first container portion so as to be in contact with said biological material; said container further comprising retention means for preventing passage of said biological material from said first container portion to said second container portion.

The retention means may be attached to a stopper adapted to be received within an opening of said first container portion. The retention means may also be in a form of a mesh located within said first container portion or in a from of a membrane. The retention means may also be part of the construction of the container, such that the first container portion may have an obstruction or narrowing in it that would prevent a solid biological material from passing therethrough.

The biological material carried by the container may comprise cells, tissues or organs (portion or whole organs). Examples of cells may include, without being limited thereto, blood cells (whole blood, blood fraction, blood constituents, etc.), semen, oocytes (ova), germ cells, zygotes, bone chips and embryos. Examples of tissues include, without being limited thereto, cartilage-containing tissue, osteochondral cartilage (such as osteochondral plugs or cylinders) excised cartilage-containing tissue and viable tissue. Examples of organs include, without being limited thereto, whole organs (optionally, including vasculature, i.e. blood vessels responsible for local supply of blood to the removed organ), e.g. whole gonadal organ; as well as fragments of organs.

According to another aspect of the present invention, there is provided a kit for freezing biological material, comprising:
(a) a device comprising:
   a cooling unit;
   a heat insulation unit, having an interface with said cooling unit and a common longitudinal axis passing through said interface transversely thereto;
   an elongated slot disposed at least in said heat insulation unit and extending along said axis, said slot comprising a first slot portion remote from said interface and a second slot portion adjacent said interface;
      wherein said first and second slot portions have respective first and second dimensions measured perpendicularly to said axis, the former being greater than the latter; and
(b) a container for accommodating therein of said biological material, said container having a longitudinal container axis and comprising a first container portion having a first dimension and a second container portion having a second dimension smaller than the first dimension, both measured perpendicularly to said container axis; the container portions being in fluid communication; said container being adapted for carrying said biological material in said first container portion, and a solution different from said material, fully filling said second container portion, which is free of said biological material, at least partially filling said first container portion so as to be in contact with said biological material;

said container further comprising retention means for preventing passage of said biological material from said first container portion to said second container portion.

The kit may further comprise a protective pouch for placing said container therein.

According to yet another aspect of the present invention, there is provided method for freezing biological material comprising:
providing a container having a first container portion and a second container portion, wherein said first portion is adapted to carry a heat insulation unit;
introducing into said container a solution and a biological material, such that said second container portion is free of said biological material and is fully filled with said solution and said first container portion is at least partially filled with said solution; and
cooling a part of said second container portion which is remote from said first container portion with a cooling means, thereby cooling the solution therein, while insulating said first container portion.

Without being bound by theory, as such knowledge may not be necessary to practice the invention, it appears that ice-seeding (ice-crystal formation) inside the sample container propagates from the second section of the sample container (being near the heat sink) towards the first section of the sample container and thus, inherently, towards the biological material located (at least mainly) in the upper part of the sample container, until the biological material is frozen.

The rate of propagation may be controlled by changing (during operation or a priori) one or more of the device's and/or the container's parameters such as the temperature differential between the cooling unit and the biological material, the distance between the front section (i.e. the tip) of the sample container and the biological material, the dimension and geometrical shape of the upper and lower parts of the sample container (and/or the corresponding dimension and geometrical shape of portions of the device that are adapted for holding the sample container or a portion thereof), the shape of the front section of the sample container etc.

Without being bound by theory, a greater the distance between the level of the cooling fluid or cooling block (in which the tip of the container is embedded) and the biological material will result in a lower cooling rate of the biological material; a greater cross sectional surface area at least at the second section (i.e. tip) of the sample container, will result in a greater cooling rate of the biological material. One or a combination of two or more of the above parameters may be taken into consideration when considering freezing of a biological sample and those versed in the art will know how to select the desired parameters based, for example, on the specific biological material to be frozen (e.g. known preferred cooling rates) and after conducting simple experimentations (e.g. measuring the temperature change during freezing and/or testing post thaw viability).

The construction of the device disclosed herein permits a temperature change front that propagates from the front section of the sample container towards the biological material carried within the upper section of the sample container. Thus, temperature change is one direction throughout the entire biological material, i.e. unidirectional temperature change.

The device and process in accordance with the invention are suitable for example for providing viable biological material following freezing with the device. The term “viable biological material” in the context of this disclosure means biological material comprising cells that are capable of surviving and maintaining their original function, provided that they are given the necessary conditions (e.g. nutrients, temperature and the like) after being thawed.

### DETAILED DESCRIPTION OF SOME NON-LIMITING EMBODIMENTS

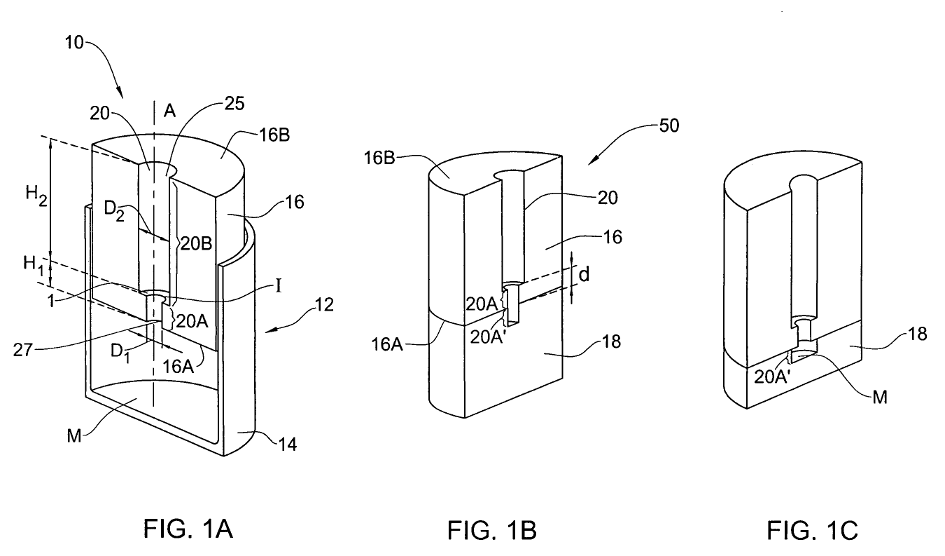

Reference is made to FIGS. 1A and 1B showing two specific but not limiting examples, respectively, of a device for changing the temperature matter within a sample container. To facilitate understanding, the same reference numbers are used for identifying components that are common in these two exemplary embodiments of the invention.

In the example of FIG. 1A, a device 10 includes a cooling unit 12 comprising a chamber 14 for carrying therein a cooling fluid medium M, and a heat insulating unit 16 having an upper surface 16B and a lower surface 16A, located (e.g. floating) on top of the fluid in the chamber 14 (or partially submerged in the fluid).

The chamber 14 is made of a thermally insulating material, for example of the type of a vacuum flask (“thermos”). The flask may be made of glass, metal or plastic with double, spaced apart walls, the region between the inner and outer walls being evacuated of air or filled with appropriate, heat-insulating plastic substance, as will be appreciated by those versed in the art.

The heat insulating unit 16 may either be fixed in place with respect to chamber 14 (e.g. by hooks or flaps that allow the heat insulating unit to hang on top of the cooling fluid medium M contained within chamber 14) or is made of material that has a specific gravity lower than that of the cooling medium M, thereby resulting in the floatation of the insulating unit 16 on top of the said cooling fluid medium M. The heat insulating unit 16 may comprise any type of insulation known per se, such as, without being limited thereto Styrofoam, cellulose wool, ceramic foams, polyethylene, vacuum.

The cooling medium may be any fluid including liquid or gas. For the purpose of freezing a sample, the medium, being a cooling medium may comprise, without being limited thereto, liquid nitrogen (LN), liquid helium or other fluid, the temperature of which is or may be controlled to be below the freezing temperature of the material to be frozen by the device.

According to some embodiments of the invention, the cooling medium M is a liquid, and the insulating unit 16 is constructed such that it would float on the cooling medium. This allows for continued contact between a tip 72 of a sample container 70 (as will be further explained) and the cooling medium, even if a portion of the cooling medium evaporates. As the level of the liquid reduces, the insulating unit 16 would move lower, such that the vertical location of the insulating unit may be used as an indicator for the need to add cooling medium M to chamber 14. The insulating unit 16 may further comprise one or more longitudinal channels and/or tubes that would allow adding cooling medium M to chamber 14 through the insulating unit and/or around it, without removing it from the chamber.

In alternative embodiments of the invention, the cooling medium M is a gas (e.g. LN vapor above liquid LN or the gaseous atmosphere above dry ice). In such cases the insulating unit 16 is fixed in place with respect to chamber 14.

The chamber 14 is typically insulated from the environment, so as to minimize temperature fluctuations (e.g. heat loss) and improve heat transfer to or from the medium carrying the biological material. The insulating material of the chamber 14 may be the same or different from that constituting the heat insulating unit 16 and may comprise glass wool, cellulose wool, ceramic foams, polyethylene, vacuum, and generally in any type of insulation known per se. The insulating material depends on the cooling medium M within the chamber. For example, if the cooling medium M is LN, a simple vacuum chamber is suitable for containing thereof. However, other insulation materials may be used, especially if circulation of the cooling medium M is required. At times, the chamber may also include a cover or be otherwise sealed, to prevent evaporation and/or contamination of the cooling medium.

The device 10 further comprises a sample slot 20 having a central axis A. While not illustrated, it is noted that the device 10 may comprise more than one sample slot 20 each having a central axis A. The sample slot 20 comprises two portions 20A and 20B. The lower portion 20A of the slot 20 has a height H₁ and diameter D₁, and the upper portion 20B of slot 20 has a height H₂ and diameter D₂, where D₁<D₂.

As shown, an interface I, defined between the upper portion 20B and lower portion 20A of slot 20, is located within the heat insulating unit 16 at a distance H₁ from the lower surface 16A of the insulating unit 16. The slot 20 has, at its top, a first opening 25 for allowing insertion of an elongated sample container into slot 20. Further, at its bottom, slot 20 defines a second opening 27, allowing a portion of the sample carrier to be immersed in the cooling medium M within container 14 and thus the direct contact between the sample carrier 70 and the cooling medium M. Alternatively, while not illustrated, insulating unit 16 may comprise several portions that may be opened (e.g. along a hinge) to allow insertion of one or more containers to respective slots, after which the insulating unit 16 is closed and at times fastened before introduction into cooling chamber 12.

In the example of FIG. 1B a device 50 is constructed generally similar to the above-described device 10. The device 50 comprises a cooling unit in a form of a cooling block 18 (e.g. a thermoconductive cooling block that may be made of a thermoconductive metal or metal alloy, for example aluminum) and a heat insulating unit 16.

The device 50 is different from the device 10 of FIG. 1A in that the cooling unit 18 (which corresponds to the cooling medium M in FIG. 1A) is formed from a solid structure. Accordingly, both units 18 and 16 are patterned to define a slot 20 configured to encase an elongated sample container, with a lowest portion 20A′ of slot 20 extending into and being encased by the cooling unit 18.

When using a solid cooling unit 18, the cooling unit may be constructed from a single unit or a plurality of sub-units together encasing the sample container. It may be also constructed from cooling units or from fluid cooling conduits. When using conduits, the conduits may be in fluid communication with a cooling fluid reservoir, typically through flow control valving means. By controlling the rate of flow and/or the temperature of the fluid that enters the conduits, the temperature of the cooling unit may be controlled. The cooling fluid transferred through the conduit will be typically liquid nitrogen.

In some embodiments, the lowest portion 20A′ of slot 20 that is encased by the cooling unit 18, as shown in FIG. 1B, is of such size that is adapted to be in thermal contact with the tip 72 of the sample container 70 when placed therein. Alternatively, said lowest portion 20A′ has a dimension measured perpendicular to the slot's axis greater than portion 20A (optionally extending deeper than the tip of the sample container to be encased in the chamber), as shown in FIG. 1C. Portion 20A′ thus forms a recess that may contain a cooling liquid medium M (FIG. 1C), that will be in thermal contact both with cooling unit 18 and the tip of the sample container. At times the use of such cooling liquid may provide the advantage of allowing easier removal of a frozen sample container from the device (e.g. if freezing should cause the container tip to expand). Further, it may provide improved thermal contact between a heat sink and the tip of the sample container during freezing (e.g. when the tip did not yet expand to an extent that allows contact thereof with the walls of cooling unit 18).

The device disclosed herein may comprise one or more temperature sensors for sensing the temperature of one or more of the cooling unit, such as cooling units 12, 18, the cooling medium M, the solution surrounding or in close proximity with the biological material, or that of the biological material per se.

The device may further comprise a control utility for controlling, inter alia, the operational steps of the device. The control utility may comprise a dedicated computer or external desktop or laptop computer or PLC (Programmable Logic Controller). It may also comprise a user interface allowing a user to control or override the pre-set operational steps of the device. In addition, in some cases, it may be desirable to provide additional information to the control utility (information such as the temperature change front propagation feedback). This information may be used for example as feedback for control of operation and also for quality assurance of the resultant temperature change of the biological material.

Furthermore, the data so collected may be stored in any form, such as digital data or printed documentation, for any use, including clinical use (e.g. for process control and verification such as to confirm that the process took place correctly, thereby indicating viability of frozen biological material for medical applications), as well as for research and development.

In addition to temperature sensors the device may include additional sensors such as:
1. One or more CCD cameras that may be used for observation of the biological material and crystals formed therein;
2. One or more temperature sensors (e.g. a thermocouple or infrared camera or detector) at one or more locations within the biological material, that may be used to record the temperature pattern at any time and the changes in temperature during operation (optionally connected to an alert to be activated in case of significant deviation from a desired temperature);
3. One or more electrical resistance (impedance) measuring units that allow detection of changes within the biological material during operation.

Ultrasound may be used to follow the freezing temperature front propagation inside the biological material. In such case an ultrasound transmitter may be used, for example within the sample container, and the propagation of the temperature front may be monitored by ultrasound readings as known in the art.

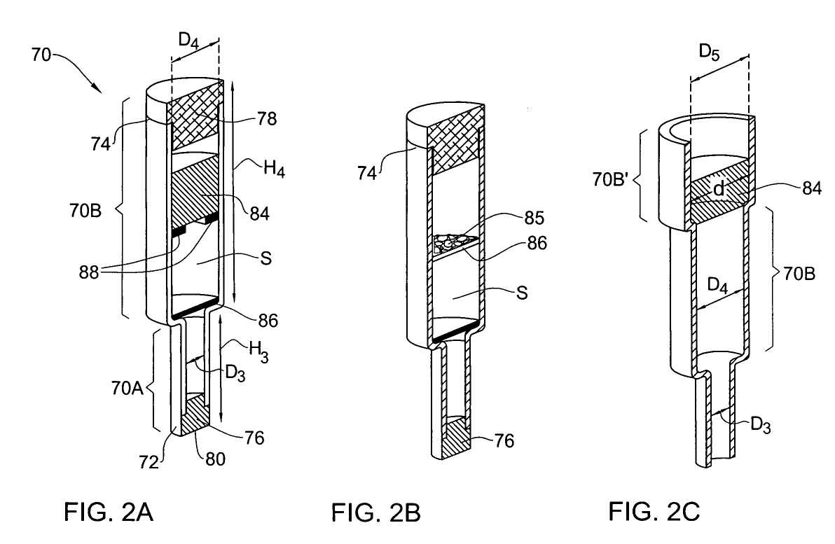

Reference is now made to FIGS. 2A to 2C, showing cross-sectional views of a non-limiting example of an elongated sample container 70 for carrying biological material therein. To facilitate understanding, the same reference numbers are used for identifying components that are common in these three exemplary embodiments of the invention.

The term “sample” as disclosed herein typically comprises biological material within a physiologically acceptable solution S. In the following description, when referring to “biological material” it includes also the physiological solution in which it is carried, unless otherwise stated.

The sample container 70 is constructed to provide adequate conditions for holding therein the biological material. It is thus made of material that withstands sterilization (using steam, gamma irradiation or other conventional methods) and during use can remain sealed (aseptic/sterile conditions can be maintained). The sample container is made of material that is compatible with the biological material and has compatibility with the cooling medium. This material may be rigid (e.g. polymers used for manufacturing test tubes) or flexible (e.g. polymers used for preparing blood bags), and should be capable of withstanding the freezing temperatures as needed for use (e.g. exposure to liquid nitrogen). Various polymers can be used for forming the sample container 70, as known in the art, and include, without being limited thereto, polytetrafluoroethylene, polystyrene, polyethylene, polypropylene or glass. A flexible container may, for example, be made of one or more layers of materials such as nylon, polyethylene, Teflon, Compton, or of materials suitable for creating a peelable and/or vacuum sealed cover, in which case a portion of the container may be sealable (e.g. welded) after filling.

The sample container 70 is further constructed so as to fit within slot 20 of the device. In the examples of FIGS. 2A and 2B, the sample container 70 has a general shape of a tube, having a lower portion 70A and an upper portion 70B. Lower portion 70A has a height H₃ and diameter D₃, and upper portion 70B has height H₄ and diameter D₄. The lower portion 70A comprises a front section 72 (at times referred to by the term “tip”).

As illustrated in FIG. 2A and 2B, sample container 70 also comprises an upper opening 74 and, optionally, also a lower opening 76 for removing the frozen biological material having a diameter essentially similar to that of the sample container 70. For example, force may be applied via the narrow lower opening 76 (e.g. pressure or using an elongated pushing member) to push frozen biological material out through the upper opening 74. The openings 74 and 76 may be hermetically closed by respective upper stopper 78 and lower stopper 80. The stoppers (plugs, corks, etc.) may be made of any material, shape and design known in the art to seal sample containers (e.g. for sealing tubes, bottles, flasks), for example, from plastic, silicon, or any other material inert to extreme temperature changes. In other words, the stoppers are made of material that they do not deform or disintegrate once in place and upon operation of the device. In some embodiments, lower opening 76 may be permanently sealed (e.g. like the tip of a conventional test tube).

Referring to FIG. 2A, the sample container 70B is adapted to encase a solid or semi-solid biological sample 84, such as cartilage or a portion of liver. In this case, the sample may be placed on top of a spacer element 86 or fixed at a location within the upper portion 70B of the sample container. In the latter case, the fixation of the biological material may be achieved by the use of a supporting member 88. The supporting member 88 may also extend from the internal surface of the upper stopper 78 to which the solid biological material is connected. For example, upper stopper 78 may comprise an extension (e.g. a noose or pocket shaped member) that is adapted to engage the solid sample in a manner that would define the sample's location without causing it significant damage or deformity. For example, in the case of an osteochondral cylinder, supporting member 88 may comprise a screw that is screwed in the bone portion of the cylinder and is attached, directly or indirectly, to upper stopper 78 (thus locating the cartilage in a desired position within the sample container without causing damage thereto). In another example, upper stopper 78 includes a recess therein, adapted to tightly engage a portion of the biological material (e.g. the bone portion of an osteochondral cylinder). The biological material may further be held within a basket or hammock, connected to a cover of the upper opening 74.

Referring to FIG. 2B, the sample container 70 may be in a form of a suspension 85, dispersed within the upper portion 70B. The biological material, being carried within a physiologically acceptable solution S, may comprise cells, tissues, as well as organs and any portion thereof.

The sample of biological material may be of any size which fits and may be encased within container 70. Thus, when referring to large sample it is meant any sample of biological material having a minimal dimension of a cross-section that is perpendicular to the longitudinal axis of the upper portion 70B of the elongated sample container 70, exceeds 0.5 cm. This minimal dimension may also exceed 1.6 cm and even 2.5 cm. The sample of biological material may be of large volume, meaning, any volume exceeding 5 mL. The volume may also exceed 12 mL and even 50 mL or more.

The biological material is maintained within a physiologically acceptable solution S contained within the internal space of the sample container 70 (filling the lower portion 70A and at least partially filling the upper portion 70B. The physiologically acceptable solution S is selected such to be compatible with the biological material and to improve the post thaw viability and/or functionality of the biological material, as determined by parameters known to those versed in the art. Examples for components included in physiologically acceptable solution S comprise cryoprotectant agents as known in the art, including, without being limited thereto, one or more of DMSO, glycerol, ethylene glycol, poly ethylene glycol, propylene glycol; sugars, such as, without being limited thereto, sucrose, dextrose, trehalose; and proteins, such as, without being limited thereto, serum proteins, albumin, fetal calf serum, of human or other source, etc; carbohydrates such as, without being limited thereto, hydroxy ethyl starch (HES), dextran, and polyphenols, such as, without being limited thereto, epigallocatechin gallate (EGCG).

As shown in FIGS. 2A-2C the sample container 70 optionally comprises a spacer element 86, such as a filter or a membrane, located at the upper portion 70B to allow retention of the biological material the upper part of the sample container, e.g. in cases where the biological material is dispersed within the physiologically acceptable solution. To this end, the spacer element 86 is constructed such that it essentially prevents passage of biological material a priori placed in the upper portion 70B above the spacer element 86 to below the spacer element and into lower portion 70A, while permitting transfer of solution S, and permitting penetration (or growth) of ice crystals formed in lower portion 70A to upper portion 70B. Spacer element 86 may be also located at the interface between the two portions 70A and 70B of the sample container 70 as illustrated in FIG. 2A, or at any other location along the upper portion 70B, as long as the biological material is prevented from passing from the upper portion 70B to the lower portion 70A. The vertical location of spacer element 86 limits the amount of biological material that may be held above the spacer element, in sample container 70. To allow a larger amount of biological material, spacer element 86 may be placed at a lower position along the container's axis, nearer the lower portion 70A. Needless to say that for a large amount of biological material, it is possible to construct a container with large dimensions. In addition, the vertical location of the spacer element may be chosen in order to locate the biological material at the portion of the sample container where a desired cooling rate will take place during freezing.

Alternatively or additionally, as shown in FIG. 2C, container 70 may be constructed with a cup-like segment 70B′ extending upwardly from upper portion 70B having a dimension D₅ measured perpendicularly to the container's axis, being greater than that of upper portion 70B. A solid or semi-solid biological material 84 having a diameter d, being smaller than or equal to D₅ but larger than D₄, may then be inserted into cup-like segment 70B′ without being able to pass to any portion of the container having a diameter smaller than d. It is to be appreciated that the transition between the portion having a diameter D₄ to upper portion 70B having a diameter D₅, may be sharp (i.e. being perpendicular to the container's axis, as shown) or may be gradual, and define an angle with said axis (not shown). Additionally, or alternatively, the walls of the cup-like segment 70B′ may be parallel to the container's axis (as illustrated in FIG. 2C), however, the walls may as well define an angle with said axis (not shown). Further, the walls of cup-like segment 70B′ may be in the form of gradually extending segments, each segment having a slightly greater diameter than the preceding one such that the segment defining (forming part of) upper opening 16B has the greatest diameter within segment 70B′.

It is noted that slot 20 in insulation unit 16 should be modified to accommodate the shape and dimension of the container or a cover or any suitable adaptor added to prevent the container's displacement in the cooling chamber.

A variety of different geometries for the sample container may be applicable in accordance with the invention. These may include different cross sectional shapes of the container, being, without being limited thereto, circular, oval, elliptic, polygonal, irregular etc. Further, since ice-seeding occurs at the front section of the sample container, various geometries are applicable for the lower part of the sample container, and in particular for the front section, which may facilitate the seeding process.

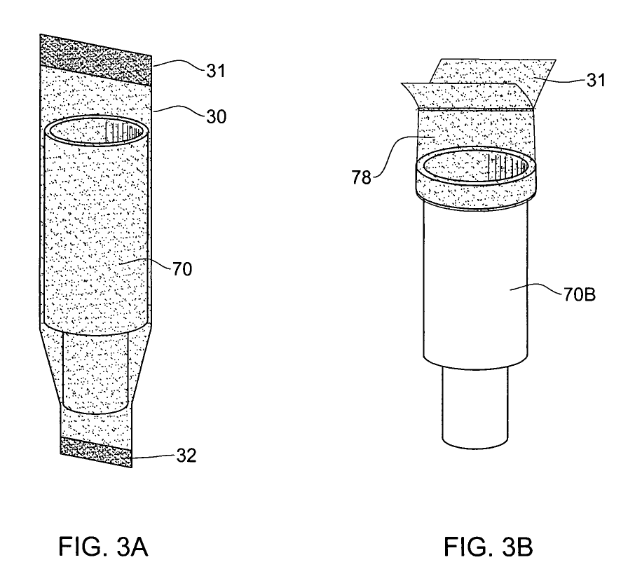

Reference is now made to FIG. 3A showing a protecting pouch 30. The sample container 70 may be placed within the protecting pouch 30 made of a sheet of sealable (e.g. fluid-sealable and/or heat sealable) material wrapped around and attached to the circumference of a portion of said container, so that the container 70 together with the biological sample is kept in sterile condition. The pouch 30 comprises an upper opening 31, through which the sample container 70 is inserted into the pouch 30. The opening then may be sealed, e.g. by welding in order to maintain the container in a closed and sterile environment. Additionally or alternatively, pouch 30 may comprise a sealable lower opening 32, similar to upper opening 31.

The pouch 30 may comprise two layers, so that an outer layer insures the sterility of an inner layer (not shown).

Referring to FIG. 3B, there is illustrated a sleeve 78 in the form of one or more sheets forming together the upper opening 31, the sheets functioning similar to pouch 30 in FIG. 3A, wrapped around and attached (or welded) to the circumference of upper portion 70B, and forming an extension thereof. After insertion of biological material into the sample container 70, the upper opening 31 of the sheets is closed, thereby hermetically sealing the container 70.

The pouch 30 may be made of materials such as nylon, polyethylene, Teflon, Compton, or other materials suitable for creating a peelable and/or vacuum sealed cover.

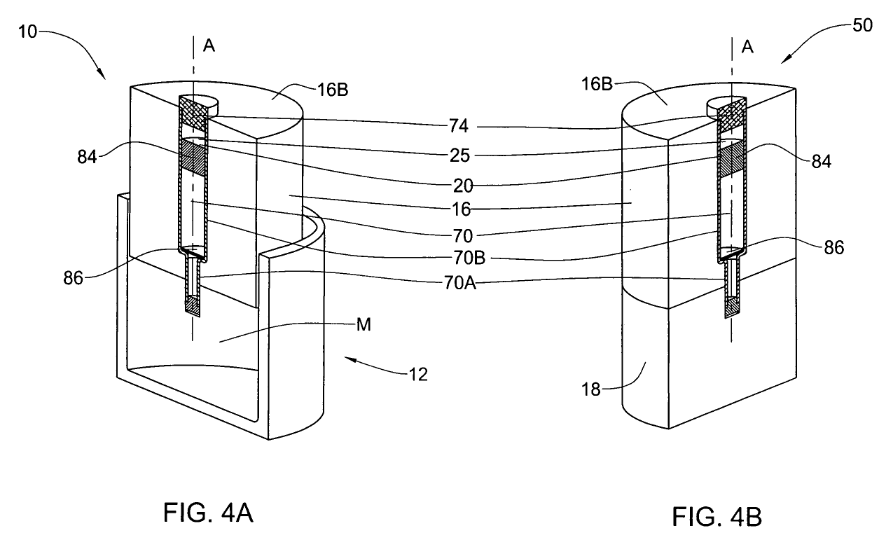

Reference is now made to FIGS. 4A and 4B showing, respectively, the devices 10 and 50 of, respectively, FIGS. 1A and 1B, in operation. To facilitate understanding, the same reference numbers are used for identifying components that are common in these two exemplary embodiments of the invention.

The sample container 70 is inserted within the slot 20, the dimensions of which correspond to the dimensions of the container 70, through the first opening 25. As shown, the sample container 70 is fitted within slot 20 such that at least a part the lower portion 70A of the sample container 70 is in thermal contact with the cooling medium M (FIG. 4A) or the cooling unit 18 (FIG. 4B).

With respect to FIG. 4B it is noted that the cooling unit 18 is configured so as to encase the corresponding part of the container's lower portion 70A. The dimensions of sample container 70 are such that once placed in the corresponding slot 20, contact between the walls of the sample container 70 and the internal walls of the slot 20 is achieved.

In the device 10 (FIG. 4A) a tight contact between the walls of the container 70 and the internal walls of the slot 20 is not required. In order to prevent any upward movement of the container 70 along the axis A, a cover (not shown) may be provided, for covering the first opening 25. In the device 50 (FIG. 4B), however, a tight contact is required between the tip of the container 70 and the lowest portion of slot 20, allowing heat to be transferred by conduction between the sample container 70 and the cooling unit 18 (if a cooling liquid is not included in a recess of the cooling unit).

The upper opening 74 of the sample container 70 ends at the upper surface 16B of the insulating unit 16, so as to ensure complete thermal isolation of the biological material within the sample container 70. In these particular embodiments, the biological material 84 is shown to be suspended over, albeit, distant from the spacer element 86.

It is essential that the temperature of the cooling unit 12, or 18 is below the freezing temperature of the biological material to be frozen. Once the sample container 70 is inserted within the respective device 10, 50, the temperature of the physiologically acceptable solution carrying the biological sample is gradually reduced. More specifically, a temperature gradient is formed within the solution. Consequently, a portion of the solution proximal to the cooling unit 12, or 18 is cooled first, and eventually freezes. The interface between a frozen portion and non-frozen portions of the solution creates a “freezing front”, which then gradually propagates in the solution as other portions of the solution gradually freeze.

The location of the biological material within the upper portion 70B is preferably adjustable so as to permit better control of its cooling. Once a location of a desired cooling rate along the sample container 70 is determined, the location of the biological sample is adjusted accordingly, i.e. within the range of the desired cooling rate. Adjustability may be achieved, inter alia, by displacing the spacer element 86 within the sample container 70, buy selecting a supporting member of a predefined length, and/or by selecting or constructing the device and sample container 70 with the appropriate proportions etc.

The cooling of the biological material is achieved by heat transferred through the solution M, from the portion of the container 70 that is remote from the cooling unit 12, or 18 (in which the biological material is accommodated, as described above) to the portion of the container 70 that is in direct contact therewith.

The temperature of the cooling unit may be essentially constant throughout the temperature change operational steps. Alternatively, the device may be constructed such that the temperature of the cooling unit is changed during the temperature change process (i.e. until the biological material is frozen). When using a fluid cooling substance for the cooling unit the temperature change may be achieved by changing the temperature of the fluid within chamber 14 in a controlled manner, using dedicated refrigerators and/or heaters.

As appreciated, the device of the invention thus provides means for changing a temperature of a biological sample, by convection, i.e. the bulk movement of thermal energy in fluids; as well as by conduction.

Once the temperature of the biological material reaches a desired temperature the sample container 70 may be removed from the slot typically for storage. This may, for example, be determined by the use of a dictated sensor or after a predetermined minimal period of time elapsed.

It is appreciated that the above discussion regarding operation of devices according to specific, non-limiting embodiments applies, mutatis mutandis, also to the method of the invention. Those skilled in the art will readily appreciate that various modifications and changes can be applied to the embodiments as hereinbefore exemplified, without departing from its scope defined in and by the appended claims.

1. A device for freezing biological material, comprising:
a cooling unit;
a heat insulation unit, having an interface with said cooling unit and a common longitudinal axis passing through said interface transversely thereto;
an elongated slot disposed at least in said heat insulation unit and extending along said axis, said slot comprising a first slot portion remote from said interface and a second slot portion adjacent said interface;
wherein said first and second slot portions have respective first and second dimensions measured perpendicularly to said slot's axis, the former being greater than the latter.

2. A device according to claim 1, wherein said slot is adapted for receiving therein a container having a first container portion for accommodating said biological material and a second container portion free of said material, such that when the container is mounted within said slot, said first container portion is disposed in the first slot portion and a portion of the second container portion is within said second slot portion.

3. A device according to claim 1, wherein the cooling unit comprises a chamber for holding a cooling fluid medium.

4. A device according to claim 3, wherein the heat insulation unit is fixed to said chamber.

5. A device according to claim 3, wherein said fluid medium is a liquid and said heat insulation unit floats on top of said liquid.

6. A device according to claim 1, wherein the cooling unit is in a form of a solid cooling block, said block comprising a recess which abuts said interface and is in fluid communication with said elongated slot disposed in said heat insulation unit.

7. A device according to claim 2, wherein the cooling unit is in a form of a solid cooling block, said block comprising a recess which abuts said interface and is in fluid communication with said elongated slot disposed in said heat insulation unit wherein said recess is adapted for having thermoconductive contact with said second container portion.

8. A device according to claim 6, wherein said recess is adapted for accommodating a cooling liquid therein.

9. A container for accommodating therein of a biological material, said container having a longitudinal container axis and comprising a first container portion having a first dimension and a second container portion having a second dimension smaller than the first dimension, both measured perpendicularly to said longitudinal container axis; the container portions being in fluid communication; said container being adapted for carrying said biological material in said first container portion, and a solution different from said material, fully filling said second container portion, which is essentially free of said biological material, and at least partially filling said first container portion so as to be in contact with said biological material; said container further comprising retention means for preventing passage of said biological material from said first container portion to said second container portion.

10. A container according to claim 9, adapted to be open at least at one end thereof.

11. A container according to claim 9, wherein said retention means are attached to a stopper adapted to be received within an opening of said first container portion.

12. A container according to claim 9, wherein said retention means are in the form of a mesh located within said first container portion.

13. A container according to claim 9, wherein said retention means is a membrane.

14. A container according to claim 9, wherein said retention means comprises a cup-like segment extending from said first portion at an opening of the container being remote from said second container portion, the cup-like segment having a dimension measured perpendicularly to the container's axis being greater than the dimension of said first portion, both measured perpendicularly to said axis.

15. A container according to claim 9, comprising a protecting pouch or a sleeve made of a sheet of sealable material wrapped around and attached to the circumference of a portion thereof forming an extension of said container.

16. A container according to claim 15, wherein said container is placed within said protecting pouch.

17. A kit for freezing biological material, comprising:
(a) a device comprising:
a cooling unit;
a heat insulation unit, having an interface with said cooling unit and a common longitudinal axis passing through said interface transversely thereto;
an elongated slot disposed at least in said heat insulation unit and extending along said axis, said slot comprising a first slot portion remote from said interface and a second slot portion adjacent said interface;
wherein said first and second slot portions have respective first and second dimensions measured perpendicularly to said axis, the former being greater than the latter; and
(b) a container for accommodating therein of said biological material, said container having a longitudinal container axis and comprising a first container portion having a first dimension and a second container portion having a second dimension smaller than the first dimension, both measured perpendicularly to said container axis; the container portions being in fluid communication; said container being adapted for carrying said biological material in said first container portion, and a solution different from said material, fully filling said second container portion, which is free of said biological material, at least partially filling said first container portion so as to be in contact with said biological material; said container further comprising retention means for preventing passage of said biological material from said first container portion to said second container portion.

18. A kit according to claim 17, further comprising a protective pouch for placing said container therein.

19. A method for freezing biological material comprising:
providing a container having a longitudinal container axis and comprising a first container portion having a first dimension and a second container portion having a second dimension smaller than the first dimension, both measured perpendicularly to said container axis; the container portions being in fluid communication;
introducing into said container a solution and a biological material, such that said second container portion is free of said biological material and is fully filled with said solution and said first container portion is at least partially filled with said solution; and
cooling a part of said second container portion which is remote from said first container portion with a cooling means, thereby cooling the solution therein, while insulating said first container portion and a remainder of said second container portion.
